(19) United States
(12) Patent Application Publication
(10) Pub. No.: US 2017/0196917 A1
(43) Pub. Date: Jul. 13, 2017

(54) AGENT FOR PREVENTION AND TREATMENT OF CHLAMYDIA INFECTION

(71) Applicant: KABUSHIKI KAISHA YAKULT HONSHA, Minato-ku (JP)

(72) Inventors: Takashi ASAHARA, Minato-ku (JP); Akira TAKAHASHI, Minato-ku (JP); Koji NOMOTO, Minato-ku (JP); Takashi KURAKAWA, Minato-ku (JP); Shoichi KADO, Minato-ku (JP); Norikatsu YUKI, Minato-ku (JP); Takuya TAKAHASHI, Minato-ku (JP)

(73) Assignee: KABUSHIKI KAISHA YAKULT HONSHA, Minato-ku (JP)

(21) Appl. No.: 15/309,551

(22) PCT Filed: May 14, 2015
(86) PCT No.: PCT/JP2015/063858

§ 371 Date: Nov. 8, 2016

(30) Foreign Application Priority Data
May 15, 2014 (JP) 2014-101200

(51) Int. Cl. A61K 35/747; A61K 9/00

(52) U.S. Cl. CPC A61K 35/747; A61K 9/0053

(57) ABSTRACT

An agent for the prevention and treatment of chlamydia infection containing Lactobacillus casei as an active ingredient which is an agent for the prevention and treatment of chlamydia infection that is highly safe and that can be administered over an extended period of time.

### TECHNICAL FIELD

The present invention relates to an agent for the prevention and treatment of chlamydia infection containing Lactobacillus casei as an active ingredient.

### BACKGROUND ART

Chlamydia infection is a disease caused by the pharyngeal or genital infection with a microorganism of Chlamydia. In particular, the infection in women causes cervicitis, pelvic inflammatory disease or the like which may result in infertility due to oviduct disorder in severe cases.

Some patients with chlamydia infection have almost no subjective symptom, and women, sometimes become pregnant without realizing chlamydia infection. Increase in the risk of abortion or premature birth in this case is a big social problem. Moreover, it is estimated that the number of patients with chlamydia infection increases especially among young people. Many of the patients do not receive any medical treatment when there is almost no subjective symptom, and it is a big social problem that the patients who do not realize the infection spread the infection by sex.

Various methods for preventing and treating chlamydia infection have been investigated so far, and for example, an antibacterial agent against a microorganism of Chlamydia has been reported (PTL 1).

However, because microorganisms of Chlamydia are intracellular parasites, there is a problem in that persistent infection is easily caused and that an antibacterial agent is not so effective. Moreover, prolonged administration is preferable to completely kill microorganism's of Chlamydia, but antibacterial agents are often not suitable for prolonged administration because antibacterial agents generally have side effects.

### CITATION LIST

### Patent Literature

PTL 1: JP-A-9-216824

### SUMMARY OF INVENTION

### Technical Problem

Accordingly, a problem to be solved by the invention is to provide an agent for the prevention and treatment of chlamydia infection and an agent for the improvement of infertility caused by chlamydia infection which are highly safe and which can be administered over an extended period of time.

### Solution to Problem

As a result of intensive investigation to solve the above problem, the present inventors have found that chlamydia infection can be prevented and/or treated by administering Lactobacillus casei. The inventors have also found that infertility caused by chlamydia infection can be improved by administering Lactobacillus casei. The invention has been thus completed.

That is, the invention relates to an agent for the prevention and treatment of chlamydia infection containing Lactobacillus casei as an active ingredient.

The invention also relates to an agent for the improvement of infertility caused by chlamydia infection containing Lactobacillus casei as an active ingredient.

Moreover, the invention relates to use of Lactobacillus casei for the manufacture of an agent for the prevention and treatment of chlamydia infection, Lactobacillus casei for the prevention and/or treatment of chlamydia infection or a method for preventing and/or treating chlamydia infection characterized by administering Lactobacillus casei.

Furthermore, the invention relates to use of Lactobacillus casei for the manufacture of an agent for the improvement of infertility caused by chlamydia infection, Lactobacillus casei for the improvement of infertility caused by chlamydia infection or a method for improving infertility caused by chlamydia infection characterized by administering Lactobacillus casei.

### Advantageous Effects of Invention

The agent for the prevention and treatment of chlamydia infection of the invention contains Lactobacillus casei as an active ingredient and is excellent because the agent can prevent and/or treat chlamydia infection through oral administration or direct administration to the infected part.

Moreover, the agent for the improvement of infertility caused by chlamydia of the invention contains Lactobacillus casei as an active ingredient and can improve infertility caused by chlamydia through oral administration or direct administration to the infected part. The term infertility in the invention includes both the inability to conceive even after a certain period of sex life with the desire to conceive and the inability of a fetus (embryo) to grow until the birth due to abortion or premature birth after conception. Thus, the agent for the improvement of infertility of the invention can improve the fertility rate of female individuals. In this regard, the fertility rate in the invention means the ratio of female individuals which have given birth normally to the female individuals which have been mated with male individuals.

### BRIEF DESCRIPTION OF DRAWINGS

FIG. 1 A figure showing the counts of cells of the chlamydia bacterium in the vagina measured in Example 1.

FIG. 2 A figure showing the fertility rates of mice calculated in Example 2.

### DESCRIPTION OF EMBODIMENTS

Lactobacillus casei which serves as the active ingredient of the agent for the prevention and treatment of chlamydia infection and the agent for the improvement of infertility of the invention (hereinafter together referred to as “the preventive and therapeutic agent of the invention”) is not particularly limited as long as it is classified as Lactobacillus casei, and those which are commercially available, those which have been deposited at a depositary authority such as ATCC, those which are newly found, mutants thereof and the like can be used. Examples of such Lactobacillus casei include Lactobacillus casei YIT9018, Lactobacillus casei YIT9029 and the like. Of the Lactobacillus casei strains, Lactobacillus casei YIT9029 is preferable. In this regard, the Lactobacillus casei strain does not have the capability of producing hydrogen peroxide and is highly safe.

Of the Lactobacillus casei strains, the present applicant already deposited Lactobacillus casei YIT9023 on May 18, 1987 at the Ministry of International Trade and Industry, the Agency of Industrial Science and Technology, the Fermentation Research Institute (the Fermentation Research Institute has become the current National Institute of Technology and Evaluation, the Patent Microorganisms Depositary and has been transferred to a new address Room 120, 2-5-8 Kaztisakamatari, Kisarazu-shi, Chiba 292-0818) as FERM BP-1366.

The applicant also deposited Lactobacillus casei YIT9018 on Nov. 14, 1984 at the Ministry of International Trade and Industry, the Agency of Industrial Science and Technology, the Fermentation Research Institute (the Fermentation Research institute has become the current National Institute of Technology and Evaluation, the patent Microorganisms Depositary and has been transferred to a new address Room 120, 2-5-8 Kazusakamatari, Kisarazu-shi, Chiba 292-0818) as PERM BP-665.

The form of Lactobacillus casei in the preventive and therapeutic agent of the invention is not particularly limited, and examples thereof include bacterial cells, pulverized bacterial cells or the like. In the case of bacterial cells, the bacterial cells may be live cells or dead cells. The Lactobacillus casei content of the preventive and therapeutic agent of the invention is not particularly limited and may be an amount corresponding to the dosage form described below. However, an example thereof in the case of live cells is 1×10⁷ to 1×10¹³ CFU. In the ease of dead cells, live cells of 1×10⁷ to 1×10¹³ CFU can be killed by heating or the like. The administration time is not particularly limited, and the agent can be administered before the infection for the purpose of prevention or administered after the infection for the purpose of treatment. However, because the risk of abortion or premature birth increases in the case of conception after the infection with chlamydia, it is preferable to administer the preventive and therapeutic agent of the invention before the conception and before the infection for the purpose of prevention. The administration period is not particularly limited, but the agent is preferably administered continuously from seven days or more before the infection when the purpose is prevention. Also, when the purpose is treatment, the agent is preferably administered continuously for three days or more after the infection. The upper limit of the administration period is not particularly limited, but an example is continuous administration for one to two weeks. The administration frequency per day is not particularly limited, either, but an example thereof is once to three times a day. When the agent is administered as the agent for the improvement of infertility, the agent can be administered in the same manner as in the case for the purpose of prevention and treatment.

The preventive and therapeutic agent of the invention should contain Lactobacillus casei as an active ingredient. Thus, the agent may consist of Lactobacillus casei or may be composed as a combination with a pharmaceutically acceptable carrier.

Examples of the pharmaceutically acceptable carrier include glucose, lactose, sucrose, starch, mannitol, dextrin, fatty acid glycerides, polyethylene glycol, hydroxyethyl starch, ethylene glycol, polyoxyethylene sorbitan fatty acid esters, amino acids, gelatin, albumin, water, a physiological saline solution and the like. According to the need, a generally used additive such as a stabilizer, a wetting agent, an emulsifier, a binder, a tonicity agent and an excipient can be suitably added.

The dosage form of the preventive and therapeutic agent of the invention is not particularly limited, but examples thereof include liquid, powder, granules, capsules, tablets, suppositories, external preparations and the like. The preventive and therapeutic agent of the invention in such a dosage form can be prepared according to a known method for preparing a preparation using a lactic acid bacterium.

The preventive and therapeutic agent of the invention obtained in this manner can be used for the prevention and/or treatment of chlamydia infection caused by a microorganism of Chlamydia such as Chlamydia trachomatis or Chlamydia muridarum.

Moreover, the agent for the improvement of infertility of the invention can improve infertility caused by chlamydia infection and improve the fertility rate.

In this regard, the preventive and therapeutic agent of the invention exerts the effects both through oral administration and through parenteral administration such as transvaginal administration, and thus the administration method is not particularly limited. However, oral administration is preferable because oral administration is easy and suitable for prolonged administration.

By adding a predetermined amount of Lactobacillus casei to a food, a food exhibiting the action of preventing and treating chlamydia infection can be produced. That is, Lactobacillus casei is used as a food additive (material) used for giving the action of preventing and treating chlamydia infection to a final product, namely a food additive composition for the prevention and/or treatment of chlamydia infection containing Lactobacillus casei. The form of Lactobacillus casei in the food additive composition of the invention is not particularly limited, and examples thereof include bacterial cells, pulverized bacterial cells or the like. In the case of bacterial cells, the bacterial cells may be live cells or dead cells. The Lactobacillus casei content of the final product to which the food additive composition of the invention has been added is not particularly limited, but an example thereof in the case of live cells is 1×10⁷ to 133 10¹³ CFU. In the case of dead cells, live cells of 1×10⁷ to 1×10¹³ CFU can be killed by heating or the like.

### EXAMPLES

The invention is explained in detail below referring to Examples, but the invention is not limited to the Examples.

### Production Example 1

### Production of Solutions of Lactobacillus casei Cells

Lactobacillus casei YIT9029 (hereinafter referred to as “LcS”) was cultured in 10 ml of MRS medium at 3° C. for 24 hours and then centrifrugally washed twice under the conditions of at 4° C. at 5,000 g for five minutes with a sucrose-phosphate gultamate buffer (hereinafter referred to as “the SPG buffer”: 75 g/L sucrose, 0.72 g/L L-glutamic acid, 6.148 g/L disodium hydrogen phosphate 12H₂O and 0.247 g/L potassium dihydrogen phosphate in distilled water, pH 7.0). The supernatant was removed, and the cells were suspended again, in 1 ml and 10 ml of the SPG buffer to produce LcS cell solutions. The LcS cell solution obtained by suspending the cells again in 1 ml of the SPG buffer was used for transvaginal administration (live LcS cell count of 1×10¹⁰ CFU/ml), and the LcS cell solution obtained by suspending the cells again in 10 ml of the SPG buffer was used for oral administration (live LcS cell count of 1×10⁹ CFU/ml). The LcS cell solutions for transvaginal administration and oral administration were heated at 100° C. for 30 minutes to produce heated dead cell solutions.

### Reference Production Example 1

### Production of Solutions of Lactobacillus johnsonii Cells

Lj cell solutions were produced in the same manner as in Production Example 1 except that Lactobacillus johnsonii YIT2019^{T} (a standard Lactobacillus johnsonii strain: hereinafter referred to as “Lj”) was used instead of LcS.

### Example 1

### Prevention/Treatment of Chlamydia Infection by Administration of LcS

(1) Preparation of Bacterium for infection

A suspension of McCoy cells (4×10⁵ cells/ml) cultured in DMEM medium containing 10% fetal calf serum was dispensed to a 24 well plate at 1 ml per well, and the cells were cultured in a CO₂ incubator at 37° C. for 24 hours. The cultured cells were infected with 1×10⁵ cells of Chlamydia muridarum ATCC VR123^{T} (hereinafter referred to as “MoPn”) per 1 ml cultured cells, and then the supernatant was removed by centrifugation. Then, DMEM medium to which cycloheximide had been added at 1 μg/ml was added, and the cells were cultured in a CO₂ incubator at 37° C. for 72 hours. The precipitates obtained by removing the MoPn-infected cells from the plate and centrifugation were suspended id the SPG buffer. The MoPn-infected cells were subjected to ultrasound treatment (28 kHz, 5 seconds, 10 times), and the supernatant was collected by centrifugation. The MoPn in the supernatant was centrifugally washed twice under the conditions of at 4° C. at 15,000 g for an hour with the SPG buffer and then suspended in the SPG buffer to produce a solution for infection (1×10⁸ cells/ml).

(2) Infection of Mice with MoPn

To regulate the estrous cycle of female mice, progesterone (luteum injection: ASKA Pharmaceutical Co., Ltd.) was subcutaneously administered at 2.5 mg per mouse. The mice whose estrous cycle had been regulated were anesthetized with Somnopentyl according to a general method. Ten microliters (MoPn cell count of 1×10⁶ cells) of the solution for infection were injected into the vagina of each mouse under anesthesia to infect the mouse with MoPn.

(3) Administration of Cell Solutions (Six Mice Per Group)

[Group with Live LcS Cell Transvaginal Administration]

Under anesthesia with Somnopentyl, 10 μl (1×10⁸ CFU) of the LcS cell solution for transvaginal administration produced in Production Example 1 was administered to each mouse every day from seven days before the infection to three days after the infection once a day in the vagina.

[Group with Dead LcS Cell Transvaginal Administration]

Under anesthesia with Somnopentyl, 10 μl of the heated dead LcS cell solution for transvaginal administration produced in Production Example 1 was administered to each mouse every day from seven days before the infection to three days after the infection once a day in the vagina. Ten microliters of the heated dead LcS cell solvation correspond to a live LcS cell count of 1×10⁶ CFU before heating.

[Group with Live Lj Cell Transvaginal Administration]

Under anesthesia with Somnopentyl, 10 μl (1×10⁸ CFU) of the Lj cell solution for transvaginal administration produced in Reference Production Example 1 was administered to each mouse every day from seven days before the infection to three days after the infection once a day in the vagina.

[Control Group with Transvaginal Administration]

Under anesthesia with Somnopentyl, 10 μl of the SPG buffer was transvaginally administered to each mouse every day from seven days before the infection to three days after the infection once a day.

[Group with Live LcS Cell Oral Administration]

A mouse was fixed with fingers, and 100 μl (1×10⁸ CFU) of the LcS cell solution for oral administration produced in Production Example 1 was orally administered to the mouse using a stomach tube every day from seven days before the infection to three days after the infection once a day.

[Group with Dead LcS Cell Oral Administration]

A mouse was fixed with fingers, and 10 μl of the heated dead LcS cell solution for oral administration produced in Production Example 1 was orally administered to the mouse using a stomach tube every day from seven days before the infection to three days after the infection once a day. A hundred microliters of the heated dead LcS cell solution correspond to a live LcS cell count of 1×10⁸ CFU before heating.

[Group with Live Lj Cell Oral Administration]

A mouse was fixed with fingers, and 100 μl (1×10⁸ CFU) of the Lj cell solution for oral administration produced in Reference Production Example 1 was orally administered to the mouse using a stomach tube every day from seven days before the infection to three days after the infection once a day.

[Control Group with Oral Administration]

A mouse was fixed with fingers, and 100 μl of the SPG buffer was orally administered to the mouse using a stomach tube every day from seven days before the infection to three days after the infection once a day.

(4) Counting of MoPn Cells

On the third day after the infection with MoPn, the vaginal tissue was collected aseptically from each mouse which had been slaughtered by bleeding under anesthesia with Somnopentyl according to a general method, and the tissue was floated on the SPG buffer and then cryopreserved at −80° C. The tissue was thawed at room temperature and homogenized, and then 200 μl of the homogenate was put into a tube containing 400 μl of RNAprotect (for quantitative RT-PCR: Qiagen) to extract the RNA. Then, the measurement was conducted by the quantitative RT-PCR method using Qiagen Onestep RT-PCR kit and using Chlamydia trachomatis- and Chlamydia muridarum-specific primers. More specifically, reverse transcription was conducted at 50° C. for 30 minutes and at 95° C. for 15 minutes, and 45 cycles each consisting of 20 seconds at 94° C., 20 seconds at 55° C. and 50 seconds at 72° C. were conducted. The nucleotide sequences of the Chlamydia trachomatis- and Chlamydia muridarum-specific primers were the

forward primer:
(SEQ ID NO: 1)
TGCATAGATAATTTGTCCTTAACTTG
and the
reverse primer:
(SEQ ID NO: 2)
CGAGATTTGACAACTAACTTACCT.

(5) Statistical Analysis

The average MoPn cell count and the standard deviation of each group were calculated. The statistical significance of an administration group compared to the control group was tested by the non-parametric Mann-Whitney's U test, and the statistical difference between groups was tested by the multiple comparison method (Bonferroni). SPSS Ver.11 (SPSS Inc.) was used as the software for the statistical analysis, and the significance level was 5% in a two-tailed test in both tests.

(6) Results

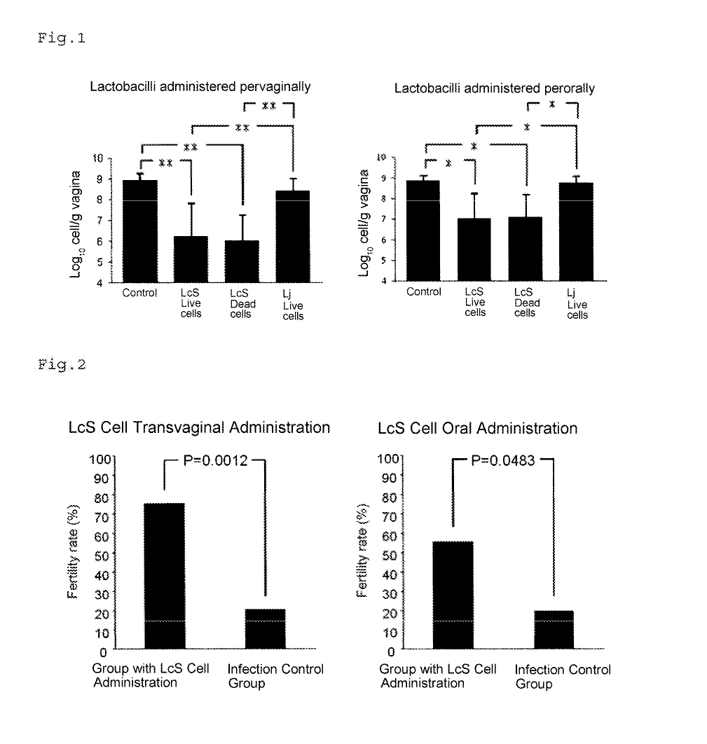

The results of the MoPn cell counts in the vagina are shown in FIG. 1.

In the group with live LcS cell transvaginal administration, the MoPn cell count in the vagina was significantly lower (P<0.01) than that of the control group with transvaginal administration. Also in the group with dead LcS cell transvaginal administration, the action of protecting against the infection which was as significant as that of the group with live LcS cell transvaginal administration was observed (P<0.01). On the other band, the action of protecting against the vaginal infection with MoPn was not observed in the group with live Lj cell transvaginal administration. In FIG. 1, * * indicates P<0.01.

Also in the group with live LcS cell oral administration, the MoPn cell count in the vagina was significantly lower (P<0.05) than that of the control group with oral administration, and the action of protecting against the vaginal infection with MoPn was observed. Moreover, in the group with dead LcS cell oral administration, the action of protecting against the infection which was as significant as that of the group with live LcS cell oral administration was observed (P<0.05). On the other hand, the action of protecting against the vaginal infection with MoPn was not observed in the group with live Lj cell oral administration. In FIG. 1, * indicates P<0.05.

### Example 2

### Improvement of Fertility Rate of Mice by LcS Administration

(1) Infection of Mice with MoPn

To regulate the estrous cycle of female mice, progesterone (luteum injection: ASKA Pharmaceutical Co., Ltd.) was subcutaneously administered at 2.5 mg per mouse. The mice whose estrous cycle had been regulated were anesthetized with Somnopentyl according to a general method. Ten microliters (MoPn cell count of 1×10⁶ cells) of the solution for infection prepared in Example 1 were injected into the vagina, of each mouse under anesthesia to infect the mouse with MoPn.

(2) Administration of Cell Solutions (20 Mice Per Group)

[Group with Live LcS Cell Transvaginal Administration]

Under anesthesia with Somnopentyl, 10 μl (1×10⁸ CFU) of the LcS cell solution for transvaginal administration produced in Production Example 1 was administered to each mouse every day from seven days before the infection to three days after the infection once a day in the vagina.

[Control Group with Transvaginal Administration]

Under anesthesia with Somnopentyl, 10 μl of the SPG buffer was transvaginally administered to each mouse every day from seven days before the infection to three days after the infection once a day.

[Group with Live LcS Cell Oral Administration]

A mouse was fixed with fingers, and 100 μl (1×10⁸ CFU) of the LcS cell solution for oral administration produced in Production Example 1 was orally administered to the mouse using a stomach tube every day from seven days before the infection to three days after the infection once a day.

[Control Group with Oral Administration]

A mouse was fixed with fingers, and 100 μl of the SPG buffer was orally administered to the mouse using a stomach tube every day from seven days before the infection to three days after the infection once a day.

(3) Mating of Mice

A female mouse on the third day after the infection with MoPn was raised in a cage with a male mouse at male 1:female 1 for five days, thereby mating the female, mouse with the uninfected male mouse. A plug (a white waxy substance attached to the vaginal opening of a female), which is an indicator of pregnancy, was checked every morning and evening from the following day of mating to the end of mating. When the plug was observed, the female individual and the male individual were separated, and whether or not the female mouse gave birth was observed under the raising condition of one mouse per case until the 32nd day after the infection.

(4) Statistical Analysis

The fertility rates were tested by the Fisher's exact test. SPSS ver.11 (SPSS inc.) was used as the software for the statistical analysis, and the significance level was 5% in a two-tailed test.

(5) Results

The fertility rates calculated from the presence or absence of birth of the mice are shown in FIG. 2. The fertility rates of the infection control groups were 20% (four out of 20 mice). On the other hand, the fertility rate of the group with LcS transvaginal administration was 75% (15 out of 20 examples), and a significant action of improving the infertility of the mice infected with MoPn was observed (P=0.0012). Moreover, also in the group with LcS oral administration, a significant action of improving the infertility of the mice infected with MoPn was observed (the fertility irate was 55% [11 out of 20 examples], P=0.0483).

### Example 3

### Change of Chlamydia Cell Count by LcS Administration

(1) Infection of Mice with MoPn

To regulate the estrous cycle of female mice, progesterone (luteum injection: ASKA Pharmaceutical Co., Ltd.) was subcutaneously administered at 2.5 mg per mouse. The mice whose estrous cycle had been regulated were anesthetized with Somnopentyl according to a general method. Ten microliters (MoPn cell count of 1×10⁶ cells) of the solution for infection prepared in Example 1 were injected into the vagina of each mouse under anesthesia to infect the mouse with MoPn.

(2) Administration of Cell Solutions (Six Mice Per Group)

[Group with Live LcS Cell Transvaginal Administration]

Under anesthesia with Somnopentyl, 10 μl (1×10⁵ CFU) of the LcS cell solution for transvaginal administration produced in Production Example 1 was administered to each mouse every day from seven days before the infection to three days after the infection once a day in the vagina.

[Control Group with Transvaginal Administration]

Under anesthesia with. Somnopentyl, 10 μl of the SPG buffer was transvaginally administered to each mouse everyday from seven days before the infection to three days after the infection once a day.

[Group with Live LcS Cell Oral Administration]

A mouse was fixed with fingers, and 100 μl (2×10⁸ CFU) of the LcS cell solution for oral administration produced in Production Example 1 was orally administered to the mouse using a stomach tube every day from seven days before the infection to three days after the infection once a day.

[Control Group with Oral Administration]

A mouse was fixed with fingers, and 100 μl of the SPG buffer was orally administered to the mouse using a stomach tube every day from seven days before the infection to three days after the infection once a day.

(3) Counting of MoPn Cells

By the same method as in Example 1, the vaginal tissue was collected from the mice on the 21st day after the infection with MoPn, and the MoPn cells in the vagina were counted.

(4) Results

In the control group with transvaginal administration, 1×10⁵ cells/g of MoPn was detected from three of the six mice also on the 21st day after the infection. On the other hand, in the group with live LcS cell transvaginal administration, the administration of the LcS cell solution had a continuous effect even though the administration was finished on the third day after the infection, and MoPn was detected from none of the mice on the 21st day after the infection.

Moreover, in the control group with oral administration, 1×10⁵ cells/g of MoPn was detected from three of the six mice also on the 21st day after the infection. On the other hand, in the group with live LcS cell oral administration, the administration of the LcS cell solution had a continuous effect even though the administration was finished on the third day after the infection, and MoPn was not detected from five of the six mice on the 21st day after the infection.

### INDUSTRIAL APPLICABILITY

The agent for the prevention and treatment of chlamydia infection of the invention can prevent and/or treat chlamydia infection. Moreover, the agent for the improvement of infertility of the invention can improve infertility caused by chlamydia infection and improve the fertility rate.

1-8. (canceled)

9. A method for treating chlamydia infection comprising administering Lactobacillus casei to a subject in used thereof.

10-11. (canceled)

12. A method for improving infertility caused by chlamydia injection, comprising administering Lactobacillus casei to a subject in need thereof.

13. The method of claim 9, wherein the Lactobacillus casei is Lactobacillus casei YIT9029 (FERM BP-1366).

14. The method of claim 9, wherein the Lactobacillus casei is administered with a pharmaceutically acceptable carrier.

15. The method of claim 14, wherein the pharmaceutically acceptable carrier is at least one selected from the group consisting of glucose, lactose, sucrose, starch, mannitol, dextrin, a fatty acid glyceride, polyethylene glycol, hydroxyethyl starch, ethylene glycol, a polyoxyethylene sorbitan fatty acid ester, an amino acid, gelatin, albumin, water, and a physiological saline solution.

16. The method of claim 9, wherein the administering of Lactobacillus casei is oral.

17. The method of claim 9, wherein the administering of Lactobacillus casei is parenteral.

18. The method of claim 12, wherein the Lactobacillus casei is Lactobacillus casei YIT9029 (FERM BP-1366).

19. The method of claim 12, wherein the Lactobacillus casei is administered with a pharmaceutically acceptable carrier.

20. The method of claim 19, wherein the pharmaceutically acceptable carrier is at least one selected from the-group consisting of glucose, lactose, sucrose, starch, mannitol dextrin, a fatty acid glyceride, polyethylene glycol, hydroxyethyl starch, ethylene glycol, a polyoxyethylene sorbitan fatty acid ester, an amino acid, gelatin, albumin, water, and a physiological saline solution.

21. The method of claim 12, wherein the administering of Lactobacillus casei is oral.

22. The method of claim 12, wherein the administering of Lactobacillus casei is parenteral


SEQUENCE LISTING (2 sequences)

SEQ ID NO: 1 - DNA, 26 bp, Artificial Sequence
  Forward Primer
tgcatagataatttgtccttaacttg

SEQ ID NO: 2 - DNA, 24 bp, Artificial Sequence
  Reverse Primer
cgagatttgacaactaacttacct